(19) United States
(12) Patent Application Publication
(10) Pub. No.: US 2018/0344615 A1
(43) Pub. Date: Dec. 6, 2018

(54) SHAMPOO COMPOSITIONS HAVING REDUCED SQUEAKINESS EFFECT, PROCESS FOR PREPARING THE SAME AND METHOD OF USE

(71) Applicant: ISP INVESTMENTS LLC, Wilmington, DE (US)

(72) Inventors: Manuel GAMEZ-GARCIA, New City, NY (US); David PETTY, Bradford (GB)

(73) Assignee: ISP INVESTMENTS LLC, Wilmington, DE (US)

(21) Appl. No.: 15/775,067

(22) PCT Filed: Nov. 17, 2016
(86) PCT No.: PCT/US2016/062504

§ 371 Date: May 10, 2018

Related U.S. Application Data
(60) Provisional application No. 62/257,788, filed on Nov. 20, 2015.

(51) Int. Cl. A61K 8/81; A61Q 5/02; A61Q 5/12

(52) U.S. Cl. CPC A61K 8/817; A61K 8/8158; A61K 2800/5426; A61Q 5/12; A61Q 5/02

(57) ABSTRACT

Disclosed herein is a hair care shampoo composition having reduced squeakiness effect comprising: (A) at least one conditioning terpolymer obtained from polymerizing: (i) about 20 wt. % to 99.9 wt. % of at least one cationic or pseudo-cationic monomer selected from the group consisting of acrylamidopropyl trimethylammonium chloride (APTAC), diallyl dimethyl ammonium chloride (DADMAC); Acryloyloxyethyltrimethylammonium chloride (AETAC); Methacrylamidopropyltrimethylammonium chloride (MAPTAC); Dimethylaminoethyl methacrylate (DMAEMA or MADAME); Methyloyloxy ethyl trimethyl ammonium chloride (METAC); Dimethylaminopropyl-methacrylamideN-(3-chloro-2-hydroxypropyl)-trimethyl-ammonium chloride (DIQUAT chloride); (ii) about 0.1 wt. % to 30 wt. % of at least one anionic monomer selected from the group consisting of (a) acrylic acid (AA), (b) acrylamidomethylpropyl sulfonate (AMPS), and/or (c) sodium methyl allyl sulfonate (SMAS); and (iii) about 0.1 wt. % to 20.0 wt. % of at least one monomer comprising at least one functionalized or unfunctionalized acryloyl moiety and at least one lactam moiety; and (B) about 0.1 wt. % to 50.0 wt. % of at least one cosmetically acceptable excipient.

### FIELD OF THE INVENTION

The present application provides a shampoo composition, and more particularly a 2-in-1 shampoo composition comprising at least one cleansing and conditioning terpolymer, process for preparing the same and method of use thereof.

### BACKGROUND OF THE INVENTION

It has been a widespread commercial practice that hair conditioners have often been separate from the shampoo composition in hair care based products. Practically, many consumers desire to have a personal hair care composition having both cleaning and conditioning functions in the same product. Such are in high demand and are known as “2-in-1” conditioning shampoo compositions, and these products comprise both cleaning and conditioning based functional components in a single product. These “2-in-1” conditioning shampoo products are capable of cleaning soiled hair and leaving a conditioner in the hair at the same time, and therefore, it is unnecessary for the consumer to use conditioner compositions subsequently after using the shampoo products.

Currently most commercial cationic polymers for use in “2 in 1” shampoos lack the ability to produce a total elimination of “squeaky-ness” induced during hair cleansing. In fact, the elimination of this unwanted sensorial attribute is the land mark for the haptics of rinse-off conditioners. “2 in 1” shampoos formulated with current commercial cationic polymers still strip the cuticle surface from its natural emolliency. Therefore, there is a need in the market for cationic polymers that truly produce the same smoothening effect that characterizes quaternaries from hair rinse-off conditioners.

Accordingly, the primary objective of the present application is to provide a 2-in-1 shampoo composition which is capable of eliminating “squeaky-ness” effect in hair and does not strip the cuticle surface of the hair. A further objective of the present application is to enhance the hair surface smoothening to a significant level.

PCT application No. 2014/071354 assigned to Hercules discloses a personal care conditioning and/or styling composition for a keratin substrate comprising: (i) about 50 wt. % to 95 wt. % of at least one cationic or pseudo-cationic monomer selected from the group consisting of acrylamidopropyl trimethylammonium chloride (APTAC) and/or Vinylpyrrolidone (VP); (ii) about 1.0 wt. % to 30 wt. % of at least one anionic monomer selected from the group consisting of (a) acrylic acid (AA), (b) acrylamidomethylpropyl sulfonate (AMPS), and/or (c) sodium methyl allyl sulfonate (SMAS); and (iii) about 0.1 wt. % to 20 wt. % of at least one hydrophobic monomer selected from the group consisting of (a) polyoxyethylene (PEG)-18-behenylether-methacrylate (BEM) (b) Lauryl-ethoxylated-methacrylate (LEM), (c) stearyl acrylate (SA), (d) Stream-10-allyl-ether, (e) Vinylcaprolactam (V-cap), and/or (f) Hydroxyethyl-pyrrolidone-methacrylate.

### SUMMARY OF THE INVENTION

The present application provides a hair care shampoo composition having reduced squeakiness effect for a keratin substrate comprising (A) at least one cleansing and conditioning terpolymer obtained from polymerizing: (i) about 20 wt. % to 99.9 wt. % of at least one cationic or pseudo-cationic monomer; (ii) about 0.1 wt. % to 30 wt. % of at least one anionic monomer; and (iii) about 0.1 wt. % to 20.0 wt. % of at least one monomer comprising at least one functionalized or unfunctionalized acryloyl moiety and at least one lactam moiety; and (B) about 0.1 wt. % to 50.0 wt. % of at least one cosmetically acceptable excipient.

A specific aspect of the present application provides a method for providing reduced squeakiness effect for hair substrate comprising contacting and washing hair substrate with an effective amount of the composition of present application.

Another aspect of the present application provides a method of cleansing and conditioning the hair using shampoo composition of present application comprises the steps of: (i) wetting the hair with required amount of water; (ii) applying an effective amount of the shampoo composition of present application to the wet hair of step (i); and rinsing off the composition from the hair using water.

Still another aspect of the present application provides a process for preparing a cleansing and conditioning terpolymer of the present application comprise polymerizing: (i) about 20 wt. % to 99.9 wt. % of at least one cationic or pseudo-cationic monomer; (ii) about 0.1 wt. % to 30 wt. % of at least one anionic monomer; and (iii) about 0.1 wt. % to 20.0 wt. % of at least one monomer comprising at least one functionalized or unfunctionalized acryloyl moiety and at least one lactam moiety.

Another aspect of the present application is to provide a significant amount of hair surface smoothening from cleansing and conditioning compositions of the present application through enabling (a) the balance of its cationic and hydrophobic composition, and (b) the ability to deposit fatty quaternaries from shampoos composition.

Yet another aspect of the present application is to provide a “2-in-1” shampoo compositions which is capable of eliminating “squeaky-ness” effect in hair and do not strip the cuticle surface from its natural emolliency. Further objective of the present application is to enhance the hair surface smoothening.

### BRIEF DESCRIPTION OF THE FIGURES

Further embodiments of the present application can be understood with the appended figures.

FIG. 1 demonstrates terpolymers of DADMAC/AMPS or ACM/PyEMA Sample.

FIG. 2 shows the molecular weight chromatogram for repeat samples IRS 7994 and 7995, 8037 and 8038.

FIGS. 3, 4 and 5 demonstrates evaluation of wet comb energy of shampoo composition comprising terpolymers.

FIG. 6 shows detangling energies of shampoo control vs. Shampoo with APTAC/AA/PyEMA.

FIG. 7 depicts reduction in squeaky effect by polymer from Shampoos.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “each independently selected from the group consisting of” means when a group appears more than once in a structure, that group may be selected independently each time it appears.

The term “about” can indicate a difference of 10 percent of the value specified. Numerical ranges as used herein are meant to include every number and subset of numbers enclosed within that range, whether particularly disclosed or not. Further, these numerical ranges should be construed as providing support for a claim directed to any number or subset of numbers in that range.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

As used herein, the words “preferred” or “preferably” and variants refer to embodiments of the application that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the application.

References herein to “one embodiment” or “one aspect” or “one version” or “one objective” of the application include one or more such embodiment, aspect, version or objective, unless the context clearly dictates otherwise.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

The term “alkyl” refers to a functionalized or unfunctionalized monovalent straight-chain, branched-chain or cyclic C₁-C₆₀ group optionally having one or more heteroatoms. Particularly, an alkyl is a C₁-C₄₅ group and more particularly, a C₁-C₃₀ group. Particular, yet non-limiting examples of alkyl groups include methyl, ethyl, n-propyl, isopropyl, n-butyl, isobutyl, tert-butyl, cyclobutyl, n-pentyl, isopentyl, cyclopentyl, n-hexyl, cyclohexyl, n-heptyl, cyclyheptyl, methylcyclohexyl, n-octyl, 2-ethylhexyl, tert-octyl, iso-norbornyl, n-dodecyl, tert-dodecyl, n-tetradecyl, n-hexadecyl, n-octadecyl, and n-eicosyl.

The term “alkylene” refers to a functionalized or unfunctionalized divalent straight-chain, branched-chain or cyclic C₁-C₄₀ group optionally having one or more heteroatoms. Particularly, an alkylene is a C₁-C₄₅ group and more particularly, a C1-C30 group. Particular, yet non-limiting examples of alkylene groups include —CH2-. —CH2-CH2-, —CH(CH3)-CH2-, —CH2-CH(CH3)-, —C(CH3)2-CH2-, —CH2-C(CH3)2-, —CH(CH3)-CH(CH3)-, C(CH3)2-C(CH3)2-, —CH2-CH2-CH2-, —CH(CH3)-CH2-CH2-, —CH2-CH(CH3)-CH2-, —CH2-CH2-CH(CH3)-, —CH2-CH2-CH2-CH2-, —CH2-CH2-CH2-CH2-CH2-, CH2-CH2-CH2-CH2-CH2-CH2-, —CH2-CH2-CH2-CH2-CH2-CH2-CH2-, cyclopropylene, cyclobutylene, cyclopentylene, cyclohexylene, and the like.

The term “halogen” refers to chloro, bromo, iodo and/or fluoro. The term “metal ion” includes alkali metal ions, alkaline earth metal ions, and transition metal ions. For example, sodium, calcium, copper and iron derived ions. The term “ammonium” includes protonated NH₃ and protonated primary, secondary, and tertiary organic amines.

The term “functionalized” refers to the state of a moiety that has one or more functional groups introduced to it by way of one or more functionalization reactions known to a person having ordinary skill in the art. Particularly, yet non-limiting examples of functionalization reactions include epoxidation, sulfonation, hydrolysis, amidation, esterification, hydroxylation, dihyroxylation, amination, ammonolysis, acylation, nitration, oxidation, dehydration, elimination, hydration, dehydrogenation, hydrogenation, acetalization, halogenation, dehydrohalogenation, Michael addition, aldol condensation, Canizzaro reaction, Mannich reaction, Clasien condensation, Suzuki coupling, and the like. Particularly, functionalization of a moiety replaces one or more hydrogens in the moiety with one or more non-hydrogen groups, for e.g., alkyl, alkoxyl, cycloalkyl, alkenyl, cycloalkenyl, alkynyl, and/or aryl groups. Particularly, yet non-limiting examples of cycloalkyl groups include cyclopentane, cyclohexane, cycloheptane, and the like. Particularly, yet non-limiting examples of alkoxy groups include methoxy, ethoxy, n-propoxy, isopropoxy, and the like. Particularly, yet non-limiting examples of aryl groups include benzenes, naphthalenes (2 rings), anthracenes (3 rings), and the like.

The term “monomer” refers to a small molecule that chemically bonds during polymerization to one or more monomers of the same or different kind to form a polymer. The term “homopolymer” refers to a polymer that consists essentially of a single monomer type. The term “non-homopolymer” refers to a polymer that comprises more than one monomer types. The term “copolymer” refers to a non-homopolymer that comprises two different monomer types.

The term “M-06” refers to N-(2-hydroxyethyl)pyrrolidone methacrylate or hydroxyethylpyrolidone methacrylate or Pyrrolidonylethyl methacrylate (PyEMA) and it has synonymously used in this application, the structure of M-06 (CAS NO: 946-25-8) is provided below:

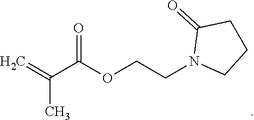

The term “alkyl (alk) acrylate” refers to an alkyl ester of an acrylic acid or an alkyl acrylic acid. The term “alkyl (alk) acrylamide” refers to an alkyl amide of an acrylic acid or an alkyl acrylic acid.

The term “acryloyl” refers to a moiety having the generic structure:

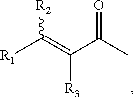

wherein each R₁, R₂, and R₃ is independently selected from the group consisting of hydrogen and functionalized and unfunctionalized alkyl, alkenyl, aryl, nitrile, formyl, carboxyl, carboxylate salt, carboxylic ester, carboxamide, halogen, thiocarboxylate, and combinations thereof

The term “each independently selected from the group consisting of” means when a group appears more than once in a structure, that group may be selected independently each time it appears.

The terms “personal care composition” and “cosmetics” refer to compositions intended for use on or in the human body, such as skin, sun, hair, oral, cosmetic, and preservative compositions, including those to alter the color and appearance of the skin and hair.

The phrase “pharmaceutically acceptable” or “cosmetically acceptable” refers to molecular entities and compositions that are generally regarded as safe. Particularly, as used herein, the term “pharmaceutically acceptable” or “cosmetically acceptable” means approved by a regulatory agency of the appropriate governmental agency or listed in the U.S. Pharmacopoeia or other generally recognized pharmacopoeia for use in animals, and more particularly in humans.

The term “turbidity” refers to the cloudiness or haziness of a fluid. The units of turbidity from a calibrated nephelometer are called Nephelometric Turbidity Units (NTU).

The term “hair care composition” refers to any composition intended for use on the human body for protection from harmful or undesirable radiation from the sun.

The term “keratin substrate” or “keratinous substrate” as used herein includes skin, nails and “keratin fibers”, and wherein the “keratin fibers” means hair on head, eyelashes, eyebrows and other mammalian bodily hair.

What is described herein is a personal care shampoo composition having reduced squeakiness effect for a keratin substrate comprising: (a) about 50 wt. % to 99.9 wt. % of at least one cationic or pseudo-cationic monomer selected from the group consisting of acrylamidopropyl trimethylammonium chloride (APTAC), diallyl dimethyl ammonium chloride (DADMAC); Acryloyloxyethyl-trimethylammonium chloride (AETAC); Methacrylamidopropyl-trimethylammonium chloride (MAPTAC); Dimethylaminoethyl methacrylate (DMAEMA or MADAME); Methyloyloxyethyl trimethyl ammonium chloride (METAC); Dimethylaminopropylmethacrylamide-N-(3-chloro-2-hydroxypropyl)-trimethylammonium chloride (DIQUAT chloride); (b) about 0.1 wt. % to 30 wt. % of at least one anionic monomer selected from the group consisting of (i) acrylic acid (AA), (ii) acrylamidomethylpropyl sulfonate (AMPS), and/or (iii) sodium methyl allyl sulfonate (SMAS); and (c) about 0.1 wt. % to 20.0 wt. % of at least one monomer comprising at least one functionalized or unfunctionalized acryloyl moiety and at least one lactam moiety (B) at least one cosmetically acceptable excipient; and optionally, at least one effective amount of personal care active ingredient.

In a preferred embodiment of the present application, the terpolymer comprising cationic or pseudo cationic monomer, anionic monomer and acryloyl moiety is capable of providing conditioning and cleaning efficacy to the keratin fibers of human and/or animals.

The preferred range of cationic or pseudo-cationic polymers, i.e., APTAC, DADMAC, MAPTAC, DMAEMA or MADAME, METAC, DIQUAT chloride for preparing a desired terpolymer of present application includes, but is not limited to, 50 wt. % to 55 wt. %; 56 wt. % to 60 wt. %; 61 wt. % to 65 wt. %; 66 wt. % to 70 wt. %; 71 wt. % to 75 wt. %; 76 wt. % to 80 wt. %; 81 wt. % to 85 wt. %; 86 wt. % to 90 wt. %; 91 wt. % to 95 wt. %. Most preferred range is 86 wt. % to 95 wt. % of the terpolymer.

The preferred range of anionic polymer, i.e., acrylic acid (AA), acrylamidomethylpropyl sulfonate (AMPS) and/or sodium methyl allyl sulfonate (SMAS) for preparing a desired terpolymer of present application includes, but is not limited to, 50 wt. % to 55 wt. %; 56 wt. % to 60 wt. %; 61 wt. % to 65 wt. %; 66 wt. % to 70 wt. %; 71 wt. % to 75 wt. %; 76 wt. % to 80 wt. %; 81 wt. % to 85 wt. %; 86 wt. % to 90 wt. %; 91 wt. % to 95 wt. %. Most preferred range is 86 wt. % to 95 wt. % of the terpolymer.

The preferred range of a monomer comprising at least one functionalized or unfunctionalized acryloyl moiety and at least one lactam moiety employed for preparing desired terpolymer of present application includes, but is not limited to, 1 wt. % to 5 wt. %; 6 wt. % to 10 wt. %; 11 wt. % to 15 wt. %; 16 wt. % to 20 wt. %; 21 wt. % to 25 wt. %; 26 wt. % to 30 wt. %. Most preferred range is 1 wt. % to 10 wt. % of the terpolymer.

The weight average molecular weight of terpolymer of the present application determined by gel permeation chromatography (GPC), is at least about 10,000, preferably about 100,000 to about 2,000,000, more preferably from about 200,000 to about 500,000 g/mol. Alternatively, viscometry can also be used to determine the average molecular weight of the present application.

A specific embodiment of the present application discloses that the compositions comprising the terpolymer of the present application is capable to eliminating the “squeaky” effect in the hair, and wherein such effect makes applicants' terpolymer unique in the range of cationic polymers for the 2-in-1 shampoo category.

According to another embodiment of the present application, it is contemplated that the smoothing effect of composition is significantly enhanced with applicants' terpolymer, and wherein, the terpolymer facilitates or allows the complexation of cationic based monomers with anionic monomer and thereby enabling the deposition of these ingredients on the hair surface.

The terpolymer of the present application has a cationic degree of substitution (Cat-DS) of greater than about 0.001 units. Other non-limiting ranges would include about 0.001 to about 5.0, about 0.2 to about 3.0, or about 0.4 to about 3.0. Further, the terpolymer of the present application has a cationic charge density in the range of from about 1 meq/g to about 8 meq/g. Preferable cationic charge density is in the range of from about 3.5 to about 7 meq/g, more preferably in the range of about 3.5 to about 5.5 meq/g.

In particular embodiments, the monomer comprising at least one functionalized or unfunctionalized acryloyl moiety and at least one lactam moiety has a structure:

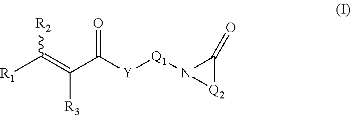

wherein each R₁ R₂ and R₃ is independently selected from the group consisting of hydrogen, halogens, functionalized and unfunctionalized C1-C4 alkyl, and

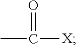

each X is independently selected from the group consisting of OR₄, OM, halogen, N(R₅)(R₆),

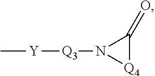

and combinations thereof; each Y is independently oxygen, NR₇ or sulfur; each R₄, R₅, R₆ and R₇ is independently selected from the group consisting of hydrogen and functionalized and unfunctionalized alkyl; each M is independently selected from the group consisting of metal ions, ammonium ions, organic ammonium cations, and combinations thereof; and each Q1, Q2, Q3, and Q4 is independently selected from the group consisting of functionalized and unfunctionalized alkylene.

Particularly, each Q1, Q2, Q3, and Q4 is independently selected from the group consisting of functionalized and unfunctionalized C1-C12 alkylene.

In one non-limiting embodiment, each R₁, R₂ and R₃ is independently selected from the group consisting of hydrogen, methyl and combinations thereof. Particularly, R₁ and R₂ are hydrogens and R₃ is hydrogen or methyl.

In another non-limiting embodiment, each R₁ and R₃ is independently hydrogen or methyl; R₂ is

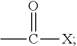

X is selected from the group consisting of OR₄, OM, halogens, and N(R5)(R6); each R4, R5, and R6 is independently selected from the group consisting of hydrogen and functionalized and unfunctionalized alkyl; and each M is independently selected from the group consisting of metal ions, ammonium ions, organic ammonium cations, and combinations thereof. Particularly, R₁ and R₃ are hydrogens and R₂ is

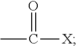

X is selected from the group consisting of OR4, OM and N(R₅)(R₆); each R₄, R₅, and R₆ is independently selected from the group consisting of hydrogen and functionalized and unfunctionalized C1-C4 alkyl; and each M is independently selected from the group consisting of metal ions, ammonium ions, organic ammonium cations, and combinations thereof.

The first polymerizable unit, defined by structure (I), can be synthesized using methods known in the art, e.g., by reaction of an N-hydroxylalkyl lactam with an acrylate, (meth)acrylate, anhydride, or similar compounds. Production methods include those described in U.S. Pat. Nos. 2,882,262; 5,523,340; 6,369,163; U.S. Patent Application Publication 2007/123673; GB 924,623; 930,668; and 1,404,989; WO 03/006569; and EP 385918. Each of the previous disclosures are hereby incorporated herein by reference in its entirety.

The lactam-containing monomers shown in structures (2)-(57) can be obtained from condensation reactions that include an N-hydroxyalkyl lactam and an unsaturated carboxylic acid, an acrylate, a (meth)acrylate, or an anhydride. Suitable N-hydroxyalkyl lactams include N-hydroxymethyl pyrrolidone and caprolactam, N-hydroxyethyl pyrrolidone and caprolactam, and N-hydroxypropyl pyrrolidone and caprolactam. Non-limiting examples of carboxylic acids that can be used include: acrylic acid, methacrylic acid, itaconic acid, crotonic acid, fumaric acid, succinic acid, and maleic acid. Similarly, acrylates and (meth)acrylates include (without limitation) methyl, ethyl, butyl, octyl, ethyl hexyl acrylates and their (meth)acrylate analogues. Representative anhydrides include formic anhydride, succinic anhydride, maleic anhydride and acetic anhydride.

In particular embodiments, the monomer having at least one functionalized or unfunctionalized acryloyl moiety and at least one lactam moiety has a structure selected from the group consisting of:

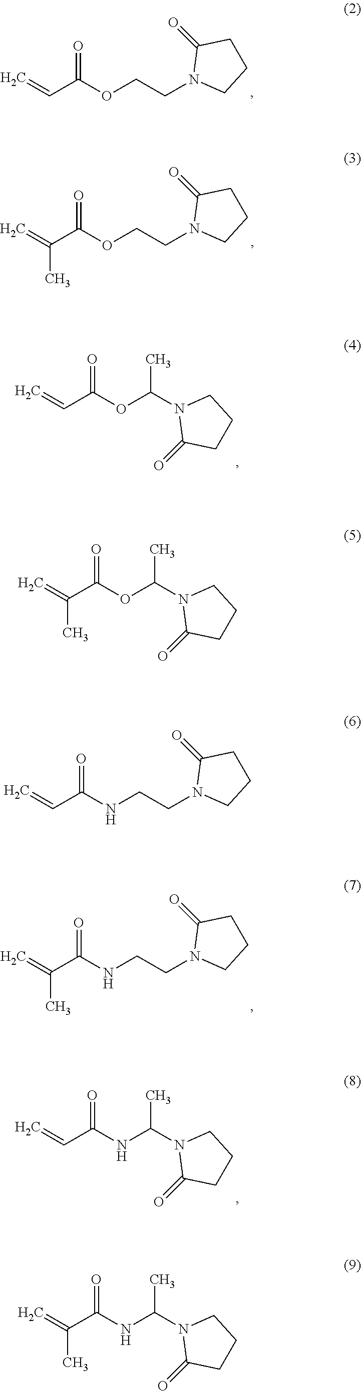

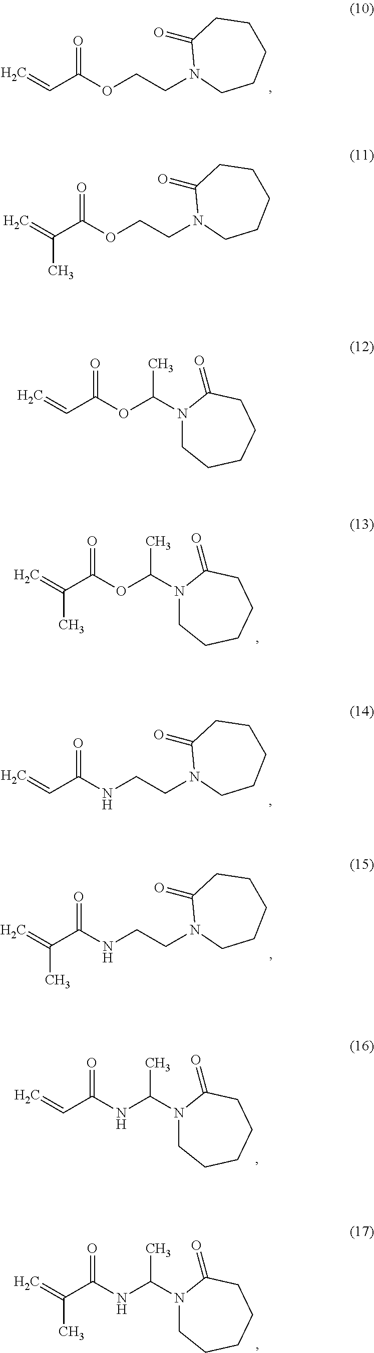

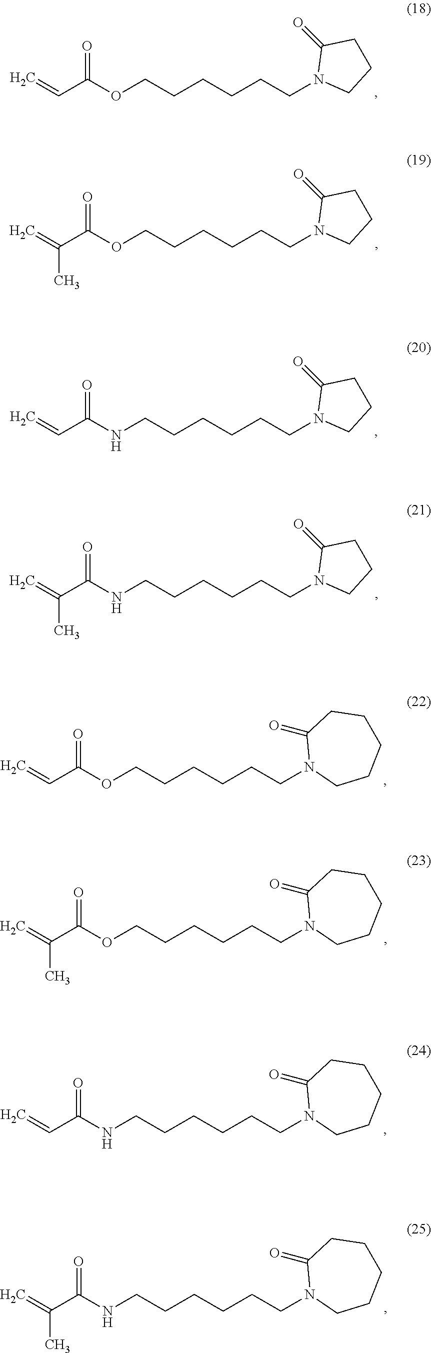

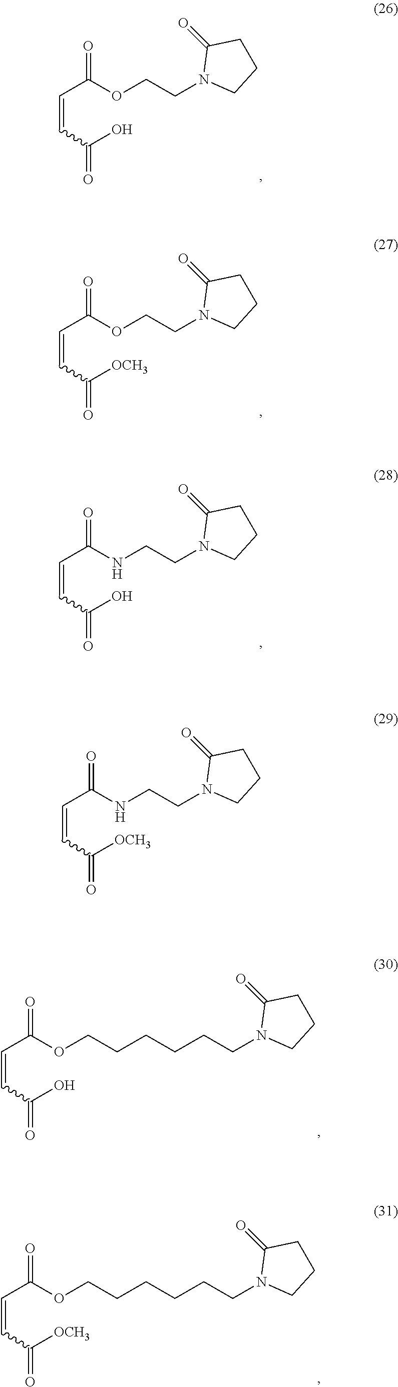 [SMILES: O=C(O)C=CC(=O)OCCN1CCCC1=O ; COC(=O)C=CC(=O)OCCN1CCCC1=O ; O=C(O)C=CC(=O)NCCN1CCCC1=O ; COC(=O)C=CC(=O)NCCN1CCCC1=O ; O=C(O)C=CC(=O)OCCCCCCN1CCCC1=O ; COC(=O)C=CC(=O)OCCCCCCN1CCCC1=O]

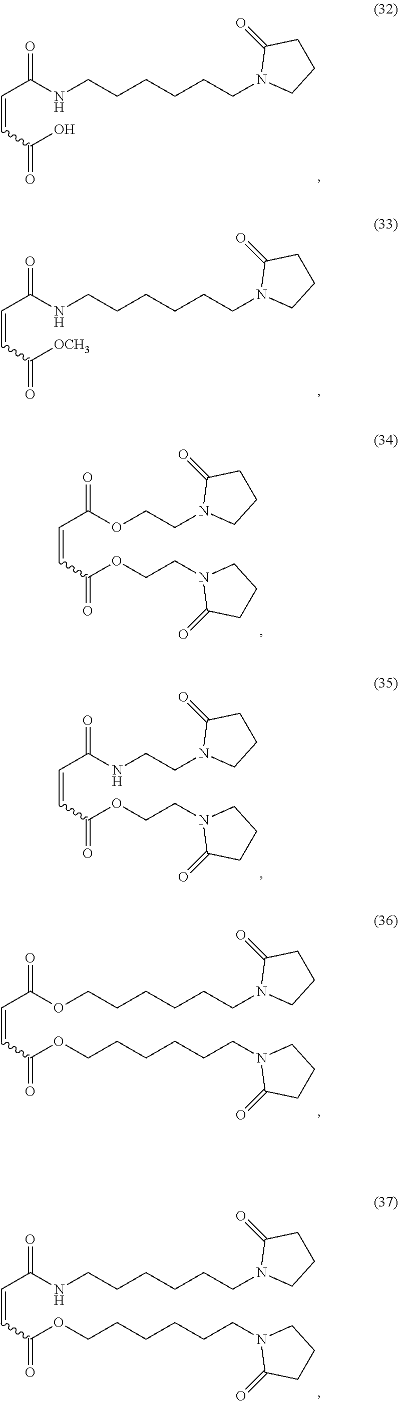 [SMILES: O=C(O)C=CC(=O)NCCCCCCN1CCCC1=O ; COC(=O)C=CC(=O)NCCCCCCN1CCCC1=O ; O=C(C=CC(=O)OCCN1CCCC1=O)OCCN1CCCC1=O ; O=C(C=CC(=O)OCCN1CCCC1=O)NCCN1CCCC1=O ; O=C(C=CC(=O)OCCCCCCN1CCCC1=O)OCCCCCCN1CCCC1=O ; O=C(C=CC(=O)OCCCCCCN1CCCC1=O)NCCCCCCN1CCCC1=O]

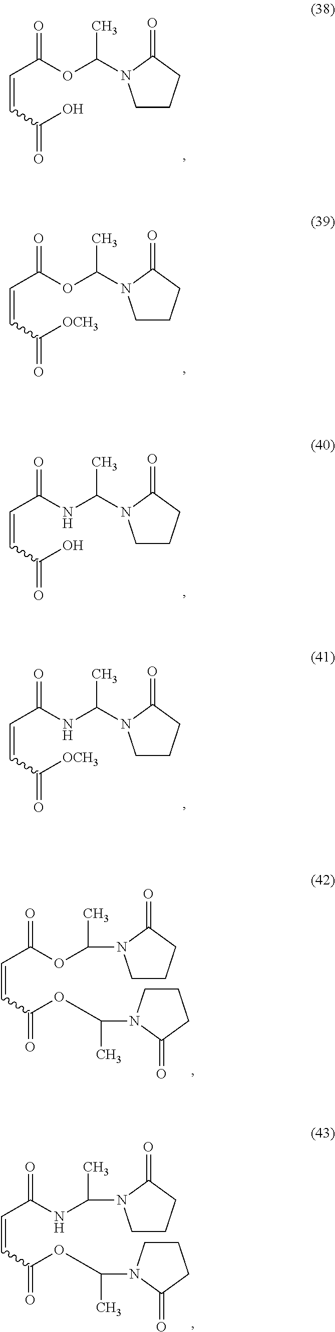 [SMILES: CC(OC(=O)C=CC(=O)O)N1CCCC1=O ; COC(=O)C=CC(=O)OC(C)N1CCCC1=O ; CC(NC(=O)C=CC(=O)O)N1CCCC1=O ; COC(=O)C=CC(=O)NC(C)N1CCCC1=O ; CC(OC(=O)C=CC(=O)OC(C)N1CCCC1=O)N1CCCC1=O ; CC(NC(=O)C=CC(=O)OC(C)N1CCCC1=O)N1CCCC1=O]

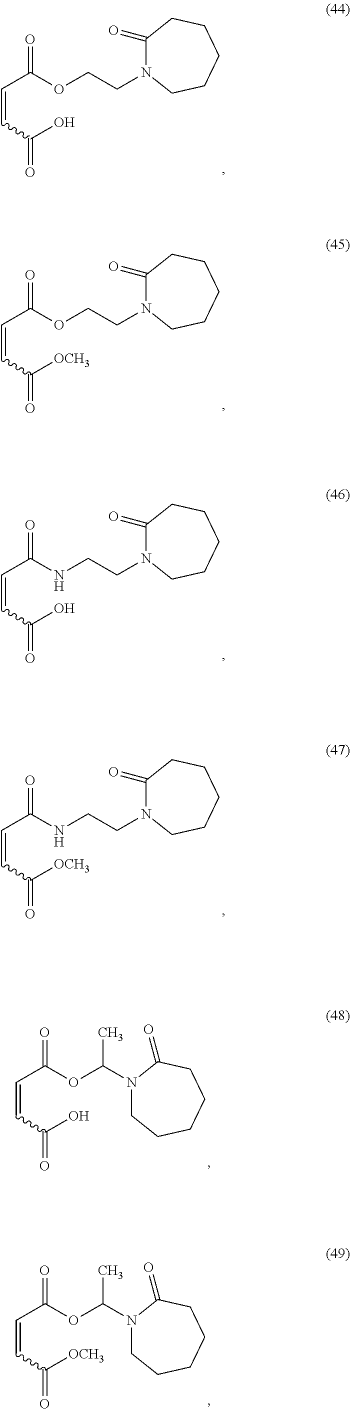 [SMILES: O=C(O)C=CC(=O)OCCN1CCCCCC1=O ; COC(=O)C=CC(=O)OCCN1CCCCCC1=O ; O=C(O)C=CC(=O)NCCN1CCCCCC1=O ; COC(=O)C=CC(=O)NCCN1CCCCCC1=O ; CC(OC(=O)C=CC(=O)O)N1CCCCCC1=O ; COC(=O)C=CC(=O)OC(C)N1CCCCCC1=O]

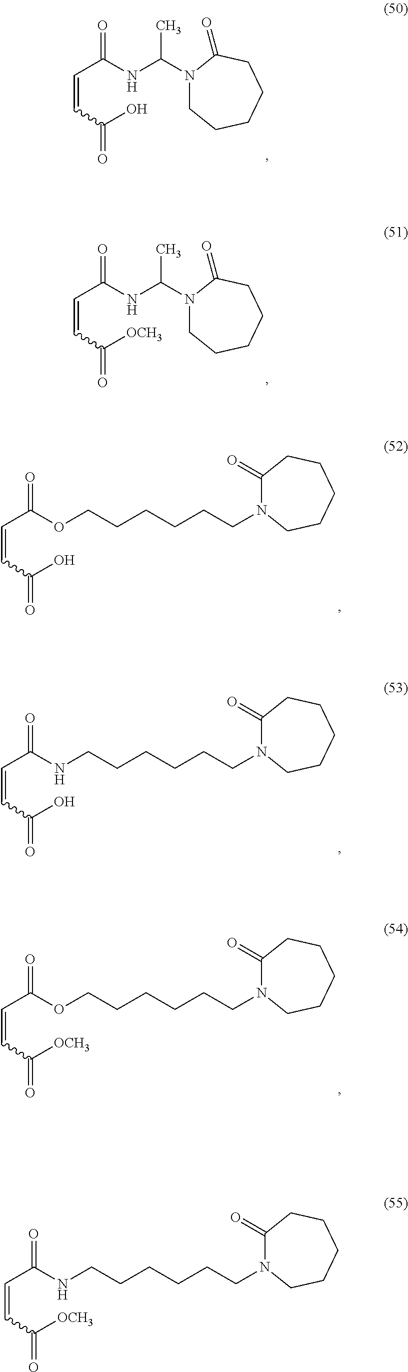 [SMILES: CC(NC(=O)C=CC(=O)O)N1CCCCCC1=O ; COC(=O)C=CC(=O)NC(C)N1CCCCCC1=O ; O=C(O)C=CC(=O)OCCCCCCN1CCCCCC1=O ; O=C(O)C=CC(=O)NCCCCCCN1CCCCCC1=O ; COC(=O)C=CC(=O)OCCCCCCN1CCCCCC1=O ; COC(=O)C=CC(=O)NCCCCCCN1CCCCCC1=O]

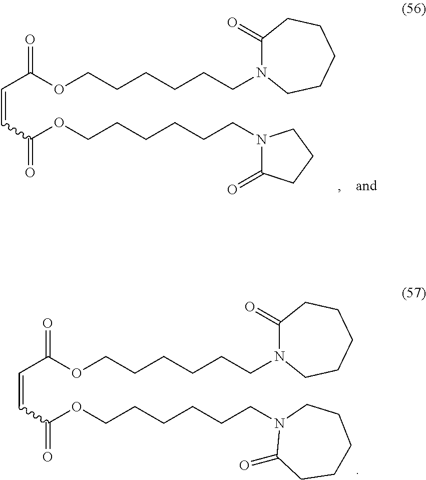 [SMILES: O=C(C=CC(=O)OCCCCCCN1CCCC1=O)OCCCCCCN1CCCCCC1=O ; O=C(C=CC(=O)OCCCCCCN1CCCCCC1=O)OCCCCCCN1CCCCCC1=O]

Other suitable examples of can be found in WO 2011/063208, the disclosure of which is hereby incorporated herein by reference in its entirety.

In a particular embodiment, the preferred functionalized or unfunctionalized acryloyl moiety and at least one lactam moiety according to the application can be selected from the following specific structures selected from the group consisting of:

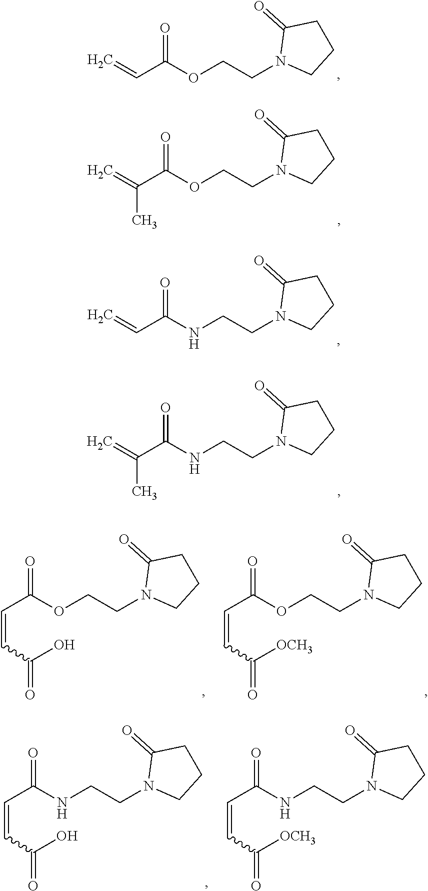

and combinations thereof

Most particularly, the terpolymer comprises functionalized or unfunctionalized acryloyl moiety and at least one lactam moiety repeating units derived from at least: about 15 to about 25 percent by weight of the polymer of at least one monomer having a structure selected from the group consisting of:

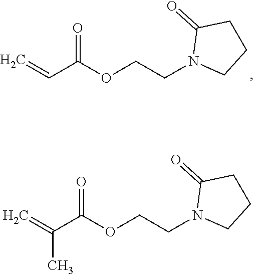

and combinations thereof,

In particular embodiments, the hair care 2-in-1 or 3-in-1 shampoo compositions having reduced squeakiness effect has a structure selected from the group consisting of:

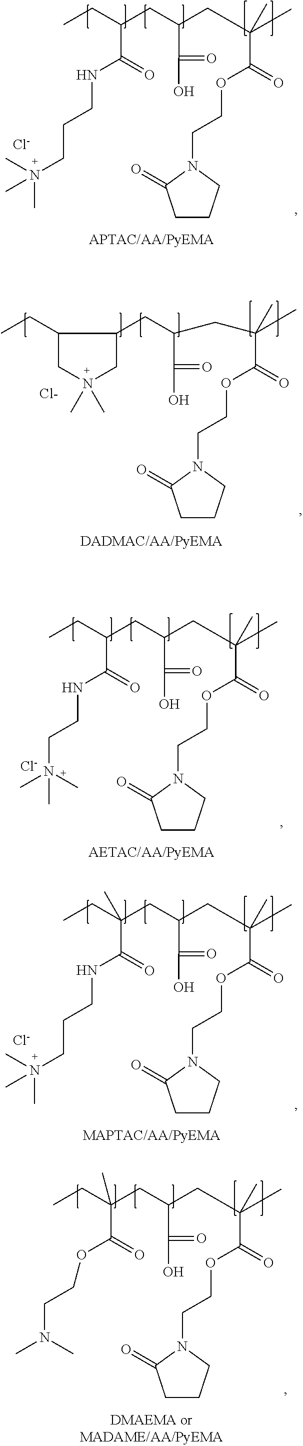

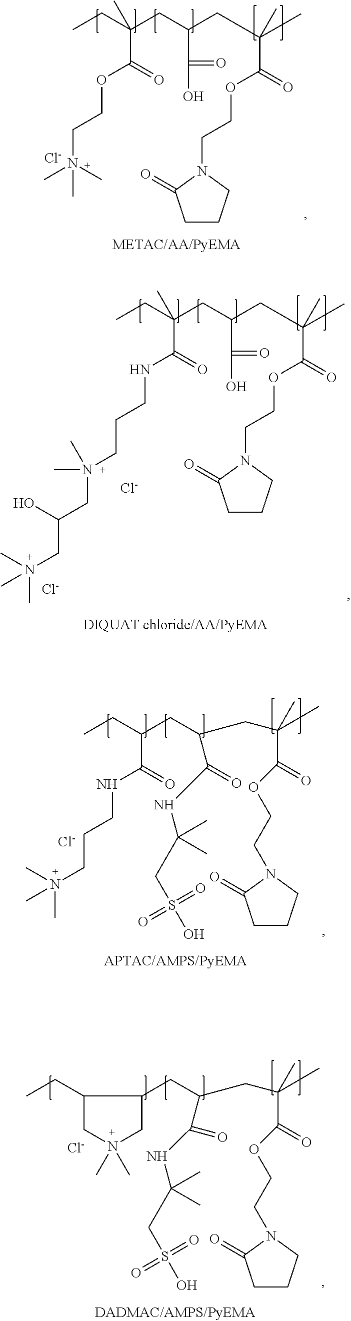

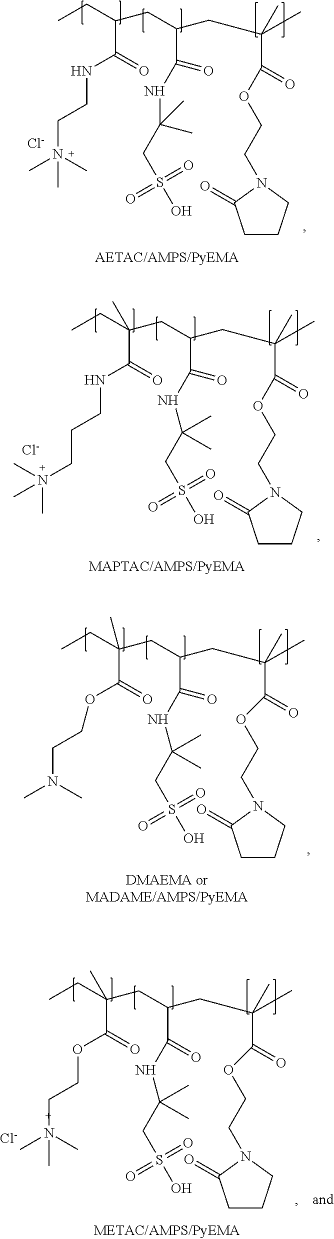

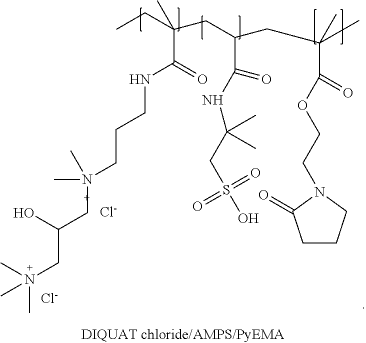

wherein each monomer is present in an independently selected value ranging from about 0.1 to about 99.9 percent by weight of the polymer, with the proviso that the sum of cationic or pseudo-cationic monomer and functionalized or unfunctionalized acryloyl moiety for each polymer equals 100 weight percent.

In a particular embodiment, the terpolymer (inventive polymer) according to the application further comprises repeating units derived from at least one crosslinker. Non limiting examples of crosslinkers include divinyl ethers.

In a particular embodiment, the terpolymer according to the present application is selected from the group consisting of acrylamidopropyl trimethylammonium chloride/N-Vinylcaprolactam/PyEMA, Acryloyloxyethyltrimethylammonium chloride (AETAC)/N-Vinylcaprolactam/PyEMA, diallyl dimethyl ammonium chloride (DADMAC)/N-Vinylcaprolactam/PyEMA, Methacrylamidopropyltrimethylammonium chloride (MAPTAC)/N-Vinylcaprolactam/PyEMA, Dimethylaminoethyl methacrylate (DMAEMA or MADAME)/N-Vinylcaprolactam/PyEMA, Methyloyloxyethyl trimethyl ammonium chloride (METAC)/N-Vinylcaprolactam/PyEMA; DimethylaminopropylmethacrylamideN-(3-chloro-2-hydroxypropyl)trimethylammonium chloride, and/or (DIQUAT chloride)/N-Vinylcaprolactam/PyEMA.

The terpolymer compositions according to the application can be used as such or formulated with other ingredient(s) resulting in various product forms.

Particularly, yet non-limiting examples of personal care compositions include hair care compositions, sun care compositions, face care compositions, lip care compositions, eye care compositions, skin care compositions, after-sun compositions, body care compositions, nail care compositions, anti-aging compositions, insect repellants, oral care compositions, deodorant compositions, conditioning compositions, color cosmetic compositions, color-protection compositions, self-tanning compositions, and foot care compositions.

The personal care composition of the present application can be a hair care “2-in-1” or “3-in-1” shampoo composition having capability to reduce the squeakiness effect. Further, the compositions of the present application are capable of fixing or treating hair and features conditioning and/or styling properties such as detangling, wet combability, wet feel, dry combability, dry feel, sheen, static flyaway control, hydrophobicity, surface smoothening, improved deposition, no build-up, color protection, and/or curl retention.

The terpolymer of the present application can be used as a hair treating agent for addressing the following non-limiting properties of hair such as moist feel, rustle feeling, wet feel, luster, smooth feel, soft feel, suppleness, bounce, smoothness, less damage, no hardness and no squeak, feel, and smooth combing.

According to an important embodiment of the present application, the personal care composition of present application can be an appropriate product apart from shampoo including products selected from the group consisting of hair-care products, hair conditioners, 2-in-1 or 3-in-1 shampoos, leave in and rinse off conditioners, hair treatments including intensive treatments, styling and treating hair compositions, hair perming products, hair straighteners, hair relaxants, hair sprays and lacquers, permanent hair dyeing systems, hair styling mousses, hair gels, semi-permanent hair dyeing systems, temporary hair dyeing systems, hair bleaching agents, permanent hair wave systems, hair setting formulations, non-coloring hair preparations, hair-frizz-control gels, hair leave-in conditioners, hair pomades, hair de-tangling products, hair fixatives, hair conditioning mists, hair care pump sprays and other non-aerosol sprays, skin-care products, hair cuticle coats, skin care moisturizing mists, skin wipes, pore skin wipes, pore cleaners, blemish reducers, skin exfoliators, skin desquamation enhancers, skin towelettes, skin protection ointments, skin powders, skin pads, paste masks and muds, face masks, facial cleansing products, anti-acne preparations, bath products, shower products, liquid soaps, bar soaps, body oils, body lotions, body gels, body and hand preparations, face and body washes, bath salts, bath and body milks, foam baths, synthetic and non-synthetic soap bars, hand liquids, shaving lotions, shaving and aftershave preparations, pre-shaves and pre-electric shaves, nail varnishes, nail polish, nail polish remover, nail creams and lotions, cuticle softeners, nail conditioners, eye shadows, mascaras, eye liners, eye shadows, blushes, makeup, eye shadow sticks, baby lotions, baby baths and shampoos, baby conditioners, fragrances and/or odoriferous ingredients consisting preparations, dentifrices, deodorizing and antiperspirant preparations, decorative preparations, light protection formulations, treatment creams, lipsticks, dry and moist make-up, rouge, powders, depilatory agents, sun care products, compositions comprising UV blockers or UV protectors, anti-aging products, foundations, face powders, moisturizing preparations, tanning preparations, nose strips, make-up removers, cold creams, mousses, shower gels, personal care rinse-off products, gels, scrubbing cleansers, astringents, lip balms, lip glosses, anhydrous creams and lotions, oil/water, water/oil, multiple and macro and micro emulsions, water-resistant creams and lotions, mouth-washes, massage oils, toothpastes, clear gels and sticks, ointment bases, topical wound-healing products, aerosol talc, barrier sprays, vitamin, herbal-extract preparations, and/or controlled-release personal care products.

The personal care composition of present application can be formulated in several required forms according to their necessity, and the non-limiting forms include emulsion, lotion, gel, vesicle dispersion, paste, cream, solid stick, mousse, shampoo, spray, balm, wipe, milk, foam, jellies, liquid, tonics, and/or enamel.

Further, it is contemplated that the terpolymer of the present application can be employed in various fields including but not limited to a personal care composition, cementing fluid, oilfield composition, construction composition, servicing fluid, gravel packing mud, fracturing fluid, completion fluid, workover fluid, spacer fluid, drilling mud, coating composition, household, industrial and institutional composition, pharmaceutical composition, food composition, biocide, adhesive, ink, paper, polish, membrane, metal working fluid, plastic, textile, printing composition, lubricant, preservative, agrochemical, or wood-care composition. Particularly, the terpolymer composition is a personal care composition, coating composition, household, industrial and institutional composition, pharmaceutical composition, or an agricultural composition. More particularly, the terpolymer composition is a personal care shampoo based composition.

The cationic polymers that can be used along with hair care shampoo composition of the present application are those known to improve the cosmetic properties of hair such as eliminating “squeaky-ness” effect. The expression “cationic polymer” or “cationic monomer” as used herein, indicates any polymer containing cationic groups and/or ionizable groups in cationic groups. The cationic polymers used generally have a molecular weight the average number of which falls between about 500 and 5,000,000 and preferably between 1000 and 3,000,000. The preferred cationic polymers are chosen from among those containing units including primary, secondary, tertiary, and/or quaternary amine groups that may either form part of the main polymer chain or a side chain. Useful cationic polymers include known polyamine, polyaminoamide, and quaternary polyammonium types of polymers, such as:

(1) Homopolymers and copolymers derived from acrylic or methacrylic esters or amides. The copolymers can contain one or more units derived from acrylamides, methacrylamides, diacetone acrylamides, acrylamides and methacrylamides, acrylic or methacrylic acids or their esters, vinyllactams such as vinyl pyrrolidone or vinyl caprolactam, and vinyl esters. Specific examples include: copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyloxyethyl trimethyl ammonium methosulfate; copolymers of vinyl pyrrolidone/dialkylaminoalkyl acrylate or methacrylate, optionally quaternized; the dimethyl amino ethyl methacrylate/vinyl caprolactam/vinyl pyrrolidone terpolymers; the vinyl pyrrolidone/methacrylamidopropyl dimethyl amine copolymer; the vinyl pyrrolidone/quaternized dimethyl amino propyl methacrylamide copolymers; and the vinyl pyrrolidone/dimethylaminopropyl methacrylamide/C9-C24 alkyldimethylaminopropyl methacrylic acid quaternized terpolymers described in U.S. Pat. No. 6,207,778; (2) Derivatives of cellulose ethers containing quaternary ammonium groups; (3) Derivatives of cationic cellulose such as cellulose copolymers or derivatives of cellulose grafted with a hydrosoluble quaternary ammonium monomer, as described in U.S. Pat. No. 4,131,576; (4) Cationic polysaccharides such as described in U.S. Pat. Nos. 3,589,578 and 4,031,307; (5) Water-soluble polyamino amides prepared by polycondensation of an acid compound with a polyamine. These polyamino amides may be reticulated; (6) Derivatives of polyamino amides resulting from the condensation of polyalcoylene polyamines with polycarboxylic acids followed by alcoylation by bi-functional agents; (7) Polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347; (8) The cyclopolymers of alkyl dialyl amine or dialkyldiallyl ammonium such as the homopolymer of dimethyl diallyl ammonium chloride and copolymers of diallyl dimethyl ammonium chloride and acrylamide; (9) Quaternary diammonium polymers such as hexadimethrine chloride. Polymers of this type are described particularly in U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020 (10) Quaternary polyammonium polymers, including; (11) The quaternary polymers of vinyl pyrrolidone and vinyl imidazole; (12) Quaternary polyamines; (13) Reticulated polymers known in the art.

Suitable Polyquaternium type cationic polymers for the present application would include, but are not limited to, Polyquaternium 4 to Polyquaternium 113. Other cationic polymers that may be used within the context of the application are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyurethanes, and derivatives of chitin.

It is contemplated to employ at least one personal care active ingredient for preparing a personal care composition of the present application comprising a terpolymer and at least one cosmetically acceptable agent, wherein, the preferred personal care active ingredient of the present application would include, but is not limited to, Carnitine, BetainAminoacids, i.e., valine, glycine, arginine, allantoin, tocopherol nicotinate, niacinamide, retinyl propionate, palmitoyl-gly-his-lys, phytosterol, polyphenolic compounds, flavonoids, flavones, flavonols, isoflavone, dexpanthenol, panthenol, bisabolol, farnesol, phytantriol, salicylic acid, zinc/sodium pyridinethione salts, piroctoneolamine, selenium disulfide, tetrahydrocurcumin, glucosamine, N-acteyl glucosamine, vitamin B3, retinoids, peptides, phytosterol, dialkanoylhydroxyproline, hexamidine, salicylic acid, N-acyl amino acids, escolols, sunscreen actives, UV-A/UV-B protecting agent, UV filters, water soluble vitamins, oil soluble vitamins, hesperedin, mustard seed extract, glycyrrhizic acid, glycyrrhetinic acid, carnosine, Butylated Hydroxytoluene (BHT) and Butylated Hydroxyanisole (BHA), ergothioneine, vanillin, vanillin derivatives, diethylhexylsyrinylidene malonate, melanostatine, sterol esters, fatty acids, poly-unsaturated fatty acids, anti-fungal agents, thiol compounds, N-acetyl cysteine, glutathione, thioglycolate, (3-carotene, ubiquinone, amino acids, idebenone, dehydroacetic acid, Licohalcone A, creatine, creatinine, feverfew extract, yeast extract, beta glucans, alpha glucans, alone or in combination.

The effective amount of personal care active ingredient employed in the present application is in the range of from about 0.01 wt. % to about 10 wt. %, preferably about 0.1 wt. % to about 5.0 wt. % and more preferably in the range of 0.05 wt. % to about 3.0 wt. % of the total composition.

As used herein, the term “cosmetically acceptable excipient” means any ingredient/compound or mixture of ingredients/compounds or compositions that are typically employed to produce other desirable effects in personal care compositions.

One important embodiment of the present application provides a process for preparing a cleansing and conditioning terpolymer comprising polymerizing: (a) about 50 wt. % to 99.9 wt. % of at least one cationic or pseudo-cationic monomer selected from the group consisting of acrylamidopropyl trimethylammonium chloride (APTAC), diallyl dimethyl ammonium chloride (DADMAC); Acryloyloxyethyltrimethylammonium chloride (AETAC); Methacrylamidopropyltrimethylammonium chloride (MAPTAC); Dimethylaminoethyl methacrylate (DMAEMA or MADAME); Methyloyloxyethyl trimethyl ammonium chloride (METAC); Dimethylaminopropylmethacrylamide-N-(3-chloro-2-hydroxypropyl)-trimethylammonium chloride (DIQUAT chloride); (b) about 0.1 wt. % to 30 wt. % of at least one anionic monomer selected from the group consisting of (i) acrylic acid (AA), (ii) acrylamidomethylpropyl sulfonate (AMPS), and/or (iii) sodium methyl allyl sulfonate (SMAS); and (c) about 0.1 wt. % to 20.0 wt. % of at least one monomer comprising at least one functionalized or unfunctionalized acryloyl moiety and at least one lactam moiety, said terpolymer having a cationic degree of substitution (Cat-DS) of greater than about 0.001 units, and cationic charge density in the range of about 1 meq/g to about 6 meq/g, and wherein, the polymer is capable of providing reduced squeakiness effect.

However, examples of non-limiting excipients useful in the practice of the present application include, preservatives, antioxidants, chelating agents, sunscreen agents, proteins, amino acids, vitamins, dyes, hair coloring agents, plant extracts, plant derivatives, plant tissue extracts, plant seed extracts, plant oils, botanicals, botanical extracts, humectants, fragrances, perfumes, oils, emollients, lubricants, butters, penetrants, thickeners, viscosity modifiers, thickeners, polymers, resins, hair fixatives, film formers, surfactants, detergents, emulsifiers, opacifying agents, volatiles, propellants, liquid vehicles, carriers, salts, pH adjusting agents, neutralizing agents, buffers, hair conditioning agents, anti-static agents, anti-frizz agents, anti-dandruff agents, hair waving agents, hair straightening agents, relaxers, absorbents, fatty substances, gelling agents, moisturizers, hydrophilic or lipophilic active agent, preserving agents, fillers, dyestuffs, reducing agents, cosmetic oils, perfumes, liquid vehicles, solvents, carriers, silicones, and combinations.

The copolymers according to the application can be readily synthesized by procedures known by those skilled in the art. Non-limiting examples of which include free radical solution polymerization, dispersion polymerization, emulsion polymerization, ionic chain polymerization, living polymerization, bulk polymerization, suspension polymerization or precipitation polymerization. Particularly, the polymerization is carried out by any one of the methods disclosed in “Principles of Polymerization” 4th edition, 2004, Wiley by George Odian and is referred and disclosed herein in its entirety and described in “Decomposition Rate of Organic Free Radical Polymerization” by K. W. Dixon (section II in Polymer Handbook, volume 1, 4th edition, Wiley-Interscience, 1999), which is herein incorporated in its entirety by reference.

The copolymers and compositions according to the application can be analyzed by known techniques. Especially preferred are the techniques of ¹³C nuclear magnetic resonance (NMR) spectroscopy, gas chromatography (GC), and gel permeation chromatography (GPC) in order to decipher polymer identity, residual monomer concentrations, polymer molecular weight, and polymer molecular weight distribution.

The following examples are presented for purposes of demonstrating, but not limiting, the preparation of the polymers, and wherein, the copolymers include:
PyEMA: Pyrrolidonylethyl methacrylate (CAS NO: 946-25-8)
APTAC: Acrylamidopropyltrimethyl-ammonium chloride
DADMAC: Dimethyldiallylammonium chloride
AETAC: Acryloyloxyethyltrimethylammonium chloride
MAPTAC: Methacrylamidopropyltrimethylammonium chloride
METAC: Methyloyloxyethyl trimethyl ammonium chloride
DIQUAT chloride: Dimethylaminopropylmethacrylamide N-(3-chloro-2-hydroxypropyl) trimethyl ammonium chloride

According to a different embodiment of the present application, it provides a method of cleansing and conditioning the hair using shampoo composition of the present application comprising the steps of: (i) wetting the hair with required amount of water; (ii) applying an effective amount of the shampoo composition to the wet hair of step (i); and (iii) rinsing off the composition from the hair using water.

Another important embodiment of the present application discloses a method for providing reduced squeakiness effect for hair substrate or keratin fibers comprising (i) wetting the hair with required amount of water; (ii) applying an effective amount of the shampoo composition to the wet hair of step (i); and (iii) rinsing off the composition from the hair using water.

Further, certain aspects of the present application are illustrated in detail by way of the following examples. The examples are given herein for illustration of the application and are not intended to be limiting thereof.

### EXAMPLES

### Example 1: General Polymerization Procedure: Method for Performing Free Rise Polymerization in Solution Polymers

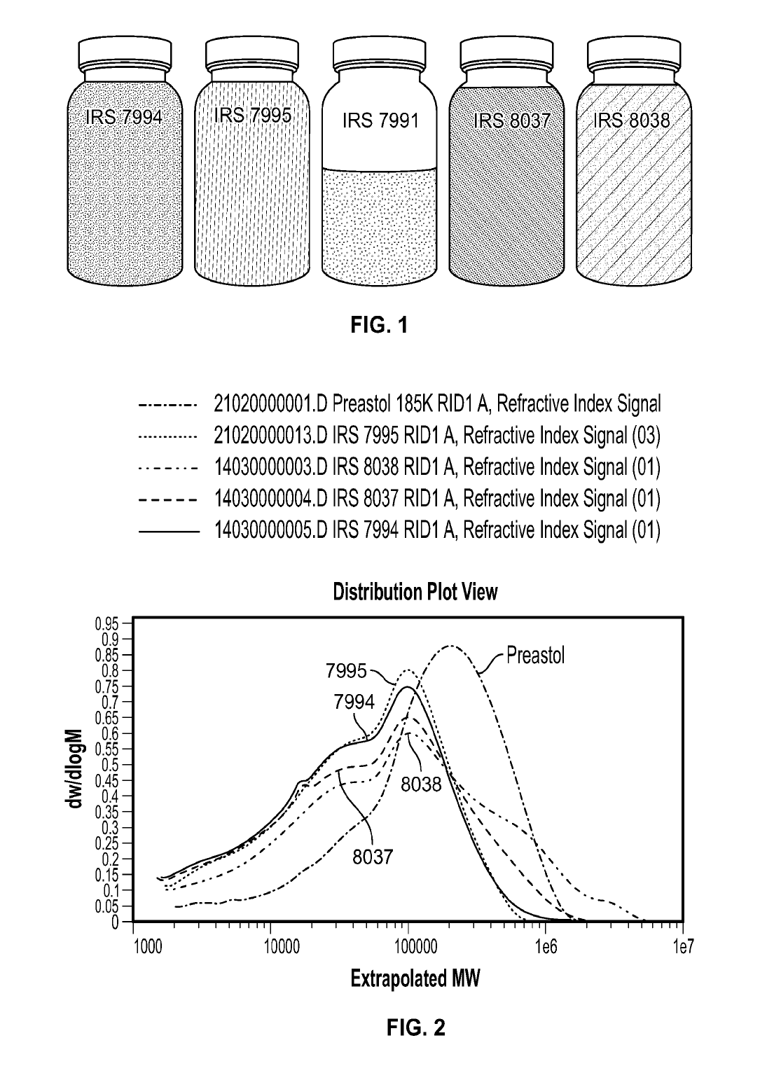

The resin pot was set up with fitted lid on the required heat source (water bath, oil bath, Huber or isomantle); The resin pot was additionally fitted with a stirrer rod, blade, stirrer gland, water cooled condenser(s), and thermometer or thermocouple and that leads to the required number of peristaltic pumps; the required monomers were weighed out and was poured into the resin pot and heated to the required temperature; The pot contents were stirred throughout the procedure and degas the pot contents with nitrogen for 30 minutes if degassing is required (N.B: The reaction charge may be aqueous and/or alcohol/solvent as dictated by the recipe). When the reaction mixture got stabilized at the required temperature, hold at the required temperature for 30 minutes prior to addition to ensure equilibrium and/or thorough degassing is achieved. The nitrogen bubbler was raised above the surface of the monomer to form a nitrogen blanket; The chain transfer reagent (if required) was added to the monomer; The thermal initiator was also added to the monomer. The reaction mixture was maintained under the set conditions until the polymerization is complete. The reaction mixture was cooled to room temperature and the pot was removed; and the polymer was characterized under the stared specifications (Table 1-3, and FIGS. 1 and 2).

TABLE 1
Terpolymers physicochemical characteristics

| | | | Solids, % | | | | | Acrylic | | |
|---|---|---|---|---|---|---|---|---|---|---|
| | Wt % | | (1 g@2 hrs | Turbidity | Viscosity | DADMAC | PYEMA | acid | | |
| Composition, wt % | ratio | pH | @110° C.) | (NTU) | cPs | residuals, % | residual | residual | Mw | Pd |
| DADMAC/AMPS/PYEMA | 87/10/3 | 6.38 | 39.60 | 6.06 | 1780 | 0.97 | — | — | 85080 | 4.65 |
| DADMAC/AMPS/PYEMA | 87/10/3 | 6.49 | 40.40 | 544 | 2150 | 0.66 | — | — | 69300 | 4.14 |
| DADMAC/AA/PYEMA | 88/8/2 | 6.00 | 45.45 | | 6780 | 0.42 | — | — | 122730 | 7.37 |
| DADMAC/ACM/PYEMA | 92/6/2 | 2.93 | 45.25 | | 143000 | 0.05 | — | — | 242510 | 10.61 |
| APTAC/AA/PYEMA | 86/8/6 | 4.39 | 22.18 | 17.68 | 96000 | 474 | 25 | 50 | 1430000 | 2.98 |
| APTAC/AA/PYEMA | 70/20/10 | 5.57 | 23.85 | 3.47 | 284000 | 447 | 27 | 2726 | 130000 | 3.2 |
| APTAC/AA/PYEMA | 70/20/10 | 5.43 | 24.51 | 5.72 | 1150 | 955 | 47 | 2368 | 1984000 | 2.39 |
| APTAC/AA/PYEMA | 85/10/5 | 5.34 | 26.22 | 4.54 | 2200 | 881 | <20 | 767 | 1819000 | 5.41 |
| APTAC/AA/PYEMA | 86/8/6 | 3.7 | 43.33 | 0 | 9200 | 3812 | 31 | 763 | 126000 | 7.1 |
| APTAC/AA/PYEMA | 94/4/2 | 4.9 | 24.08 | 0.91 | 1150 | 72 | 20 | 8 | 224948 | 2.795 |
| APTAC/AA/PYEMA | 92/6/2 | 5.27 | 23.91 | 3.14 | 1150 | 40 | 20 | 5 | 312650 | 3.047 |
| APTAC/AA/PYEMA | 96/2/2 | 4.73 | 24.64 | 2.43 | 600 | 44 | 20 | 7 | 254273 | 2.978 |
| APTAC/AA/PYEMA | 94/2/4 | 4.74 | 24.79 | 2.6 | 1200 | 87 | 20 | 12 | 319400 | 3.116 |
| APTAC/AA/PYEMA | 90/6/4 | 5.09 | 24.6 | 2.51 | 1200 | 131 | 20 | 14 | 341267 | 3.206 |
| APTAC/AA/PYEMA | 88/6/6 | 5.13 | 23.83 | 5.53 | 1100 | 78 | 20 | 8 | 322767 | 3.257 |
| APTAC/AA/PYEMA | 92/2/6 | 5.95 | 24.32 | 1.21 | 1300 | 39 | 20 | 4 | 323317 | 3.435 |
| APTAC/MAA/PYEMA | 96/2/2 | 4.36 | 23.78 | 3.44 | 1000 | 45 | — | — | 205000 | 2.27 |
| APTAC/AA/PYEMA | 96/2/2 | 4.3 | 25.27 | 4.37 | 2150 | 37 | — | — | 360000 | 3.43 |
| APTAC/AA/PYEMA | 92/6/2 | 4.28 | 42.71 | too | 41600 | 634 | 3 | — | 814000 | 8.71 |
| | | | | viscous | | | | | | |
| APTAC/AA/PYEMA | 92/6/2 | 3.93 | 45.64 | 7.88 | 10000 | 336 | 2 | — | 157000 | 2.59 |
| APTAC/AA/PYEMA | 92/6/2 | 3.9 | 44.75 | 2.71 | 2800 | 289 | 2 | — | 103000 | 2.48 |
| APTAC/AA/PYEMA | 92/6/2 | 3.82 | 42.39 | 3.66 | 1475 | 232 | 2 | — | 84000 | 2.3 |
| APTAC/AMPS/PYEMA | 85/5/10 | 3.62 | 42.21 | 30.96 | 3425 | 4 | 62 | — | 113000 | 2.19 |
| APTAC/AMPS/PYEMA | 85/5/10 | 3.72 | 42.68 | 56 | 80000 | 4 | 92 | — | 382000 | 2.41 |
| APTAC/AMPS/PYEMA | 85/5/10 | 3.58 | 42.68 | 118 | 15400 | 54 | 8 | — | 190000 | 1.9 |

TABLE 2
Synthesis of terpolymers of DADMAC/AMPS/PyEMA and DADMAC/ACM/PyEMA

| | | | | | | | Initiator |
|---|---|---|---|---|---|---|---|
| | | | | | | Initiator | feed |
| Sample | | Wt % | Experimental | Reaction | | Level | length |
| ID | Composition, wt % | ratio | conditions | Temp ° C. | Initiator | ppm/min | (hrs) |
| 7991 | DADMAC:AMPS: PYEMA | 88/10/2 | DADMAC in | 100 | APS | 100 | 4 |
| | | | pot. 100 ppm | | | | |
| | | | APS fed in | | | | |
| | | | over 4 hrs. | | | | |
| | | | AMPS/PYEMA | | | | |
| | | | fed in over 3.5 | | | | |
| | | | hr at 100° C. | | | | |
| 7994 | DADMAC:AMPS:PYEMA | 87/10/3 | DADMAC in | 100 | APS | 100 | 3.25 |
| 7995 | | | pot, AMPS and | 100 | APS | 100 | 3.25 |
| | | | PYEMA as feed | | | | |
| | | | 3 hrs. | | | | |
| | | | Reflux, APS | | | | |
| | | | (100 ppm for | | | | |
| | | | 3.25 hrs) | | | | |
| 8038 | DADMAC:ACM:PYEMA | 92/6/2 | 100 ppm APS | 100 | APS | 100 | 3.5 |
| | | | fed in over 3.5 | | | | |
| | | | hrs. | | | | |
| | | | ACM/PYEMA | | | | |
| | | | fed in over 3 | | | | |
| | | | hr at 100° C. | | | | |
| 8037 | DADMAC:ACM:PYEMA | 88/8/2 | 100 ppm APS | 100 | APS | 100 | 3.5 |
| | | | fed in over 3.5 | | | | |
| | | | hrs. | | | | |
| | | | AA/PYEMA | | | | |
| | | | fed in over 3 | | | | |
| | | | hr at 100° C. | | | | |

| | Holding | | Solids, % | | | | | |
|---|---|---|---|---|---|---|---|---|
| | time | | (1 g@2 | | Viscosity, cPs | | | |
| Sample | after | | hrs@110° | | (LVT, 12 rpm, | DADMAC | | |
| ID | polymn | pH | C.) | Turbidity | sp 3, 20° C.) | residuals, % | Mw | Pd |
| 7991 | 1 | 6.18 | 41.04 | 6.39 | 1325 | 0.98 | 82305 | 2.22 |
| 7994 | 1 | 6.38 | 39.60 | 6.06 | 1780 | 0.97 | 85080 | 4.65 |
| 7995 | 1 | 6.49 | 40.40 | — | 2150 | 0.66 | 69300 | 4.14 |
| 8038 | 2 | 2.93 | 45.25 | | 143000 | 0.05 | 242510 | 10.61 |
| 8037 | 2 | 6.00 | 45.45 | | 6780 | 0.42 | 122730 | 7.37 |

TABLE 3
Terpolymers of APTAC/AA or MAA/PyEMA

| Product | Description/Details |
|---|---|
| APTAC/AA/PyEMA (50/10/40) | pH adjusted to 4.5, 2ME and Vazo 50 |
| | shot at 55° C. |
| APTAC/AA/PyEMA (50/10/40) | No 2ME added and Vazo 50 shot at |
| | 55° C. |
| APTAC/MAA/PyEMA (96/2/2) | pH adjusted to 3.9, 2ME and Vazo 50 |
| | shot at 55° C. |

### Example 2: Shampoo Formulation

| Ingredients | Wt. % |
|---|---|
| Sodium Lauryl Sulfate-2 | 12.00 |
| (Two moles of ethoxilation) | |
| Cocamidopropylbetaine | 3.00 |
| Polymer under evaluation | 0.50 |
| Add water to complete 100% | |
| Adjust viscosity | 6000-8000 cPs with NaCl. |
| Adjust pH | ~5.5 |

### Example 3: Wet Comb Energy Evaluation

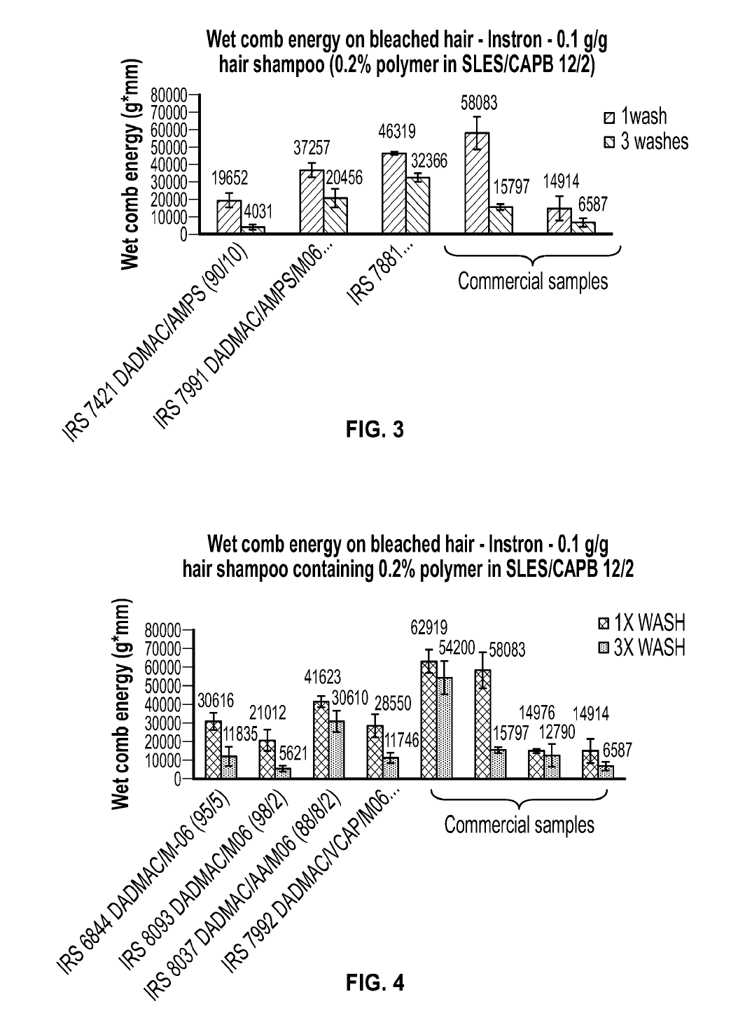

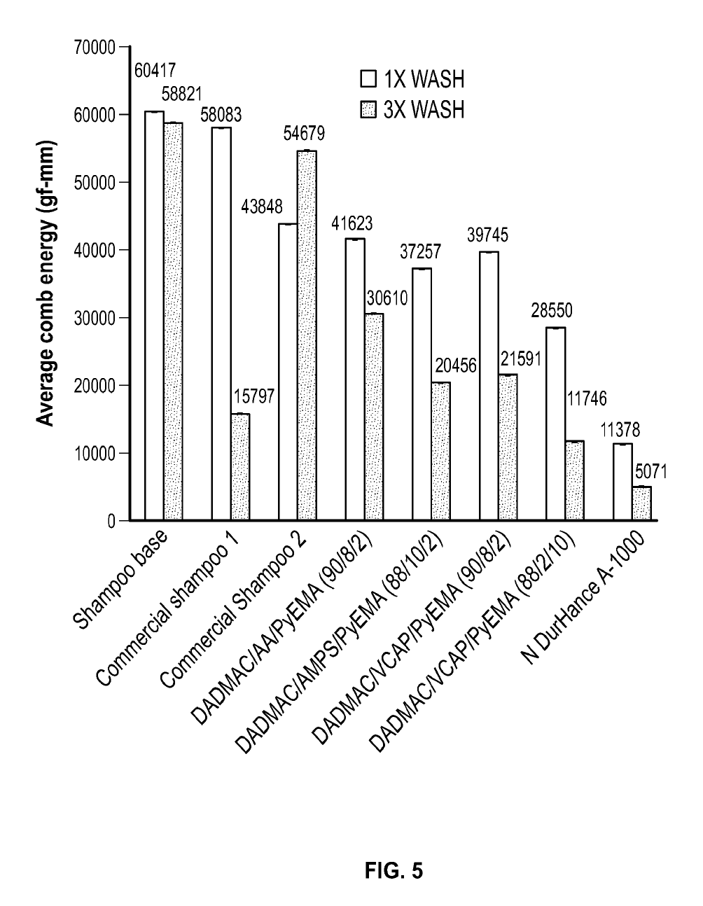

The combing measurement of the hair tresses treated with terpolymer containing shampoo was performed on Instron device. The energy needed to comb the tress was listed as gf-mm. For every measurement 3 bleached hair tresses were used and the average was calculated. To measure durable conditioning, the tress is first treated with 0.1 g/g hair shampoo, which is then rinsed off and the comb energy is measured. The tress was washed 2 more times and the comb energy was again measured. The results of wet comb energy after 1 and 3 washes are disclosed in FIG. 3, FIG. 4 and FIG. 5 for shampoo compositions comprising 0.1 wt. % of terpolymer in SLES/CAPB 12/2.

From the results of wet combability for the terpolymer shampoo compositions, it is evident that the conditioning performance of terpolymers shampoo is even better as compared to with commercial samples. The shampoos were applied on normal hair with 0.1 g grams per gram of bleached hair (1 hour bleached) and wet comb energies were measured after 1 and 3 times of washing with 0.1 grams per gram hair experimental shampoo. Terpolymers containing shampoos were outperforming commercial shampoo.

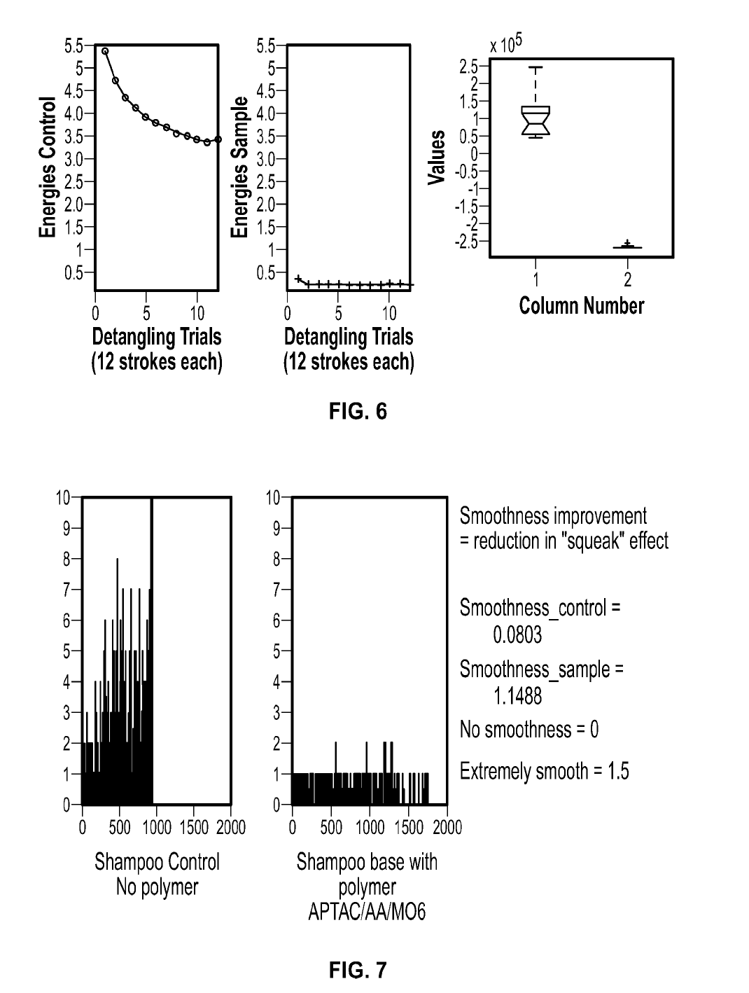

Also performed detangling studies for terpolymer of the present application for evaluating wet combability of the hair tresses using texture analyzer without manually detangling the hair, and results are duly provided in FIG. 6.

### Example 4: Method to Test Hair Tresses for a Friction Profile Equivalent to that of a Rinse-Off Conditioner (Absence of Squeaky-Ness)

Two hair tresses were cut, 1-inch-wide and 8 inches long (1.5 g) each, one was labelled as the control and the other one was labelled as sample. Around 0.5 g of a cleansing shampoo was applied to a wet hair tress that was used as control, wherein, the cleansing shampoo is the control composition and does not comprise any polymer or conditioner applied to a wet hair tress, and wherein, a simple shampoo base of 12% SLES-2+3% Cocamidopropylbetaine was recommended. Rubbed with fingers to produce foam and washed for 30 seconds, then, under running tap water (T˜30 C), the control hair tresses were rinsed for 30 seconds.

Further, the hair tress samples were washed with the shampoo containing conditioning polymer under evaluation using a washing procedure as described for control. After rinsing and while the hair tresses were still wet, the hair tresses under control were rubbed with the fingers applying a gentle force while moving from root to tip, and applied enough compression force with the fingers in such a manner so as to produce a squeaky sound.

In a similar manner, the hair tress applied with polymer samples were subjected for evaluation, in order to get familiar with the squeaky sound practice using a hair tress washed with a stripping shampoo vs a hair tress washed and treated with a rinse-off conditioner. The hair treated with the rinse-off conditioner does not produce any squeaky sound, or if it does, it should have a low intensity and high pitch (high frequency sound) (FIG. 7).

All of the articles and/or methods disclosed herein can be made and executed without undue experimentation in light of the present disclosure. While the articles and methods of the disclosed and/or claimed inventive concept(s) have been described in terms of particular aspects, it will be apparent to those of ordinary skill in the art that variations may be applied to the articles and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

What is claimed is:

1. A hair care shampoo composition having reduced squeakiness effect comprising:
A. at least one cleansing and conditioning terpolymer obtained from polymerizing:
(i) about 20 wt. % to 99.9 wt. % of at least one cationic or pseudo-cationic monomer selected from the group consisting of acrylamidopropyl trimethylammonium chloride (APTAC), diallyl dimethyl ammonium chloride (DADMAC); Acryloyloxyethyltrimethylammonium chloride (AETAC); Methacrylamidopropyltrimethylammonium chloride (MAPTAC); Dimethylaminoethyl methacrylate (DMAEMA or MADAME); Methyloyloxyethyl trimethyl ammonium chloride (METAC); DimethylaminopropylmethacrylamideN-(3-chloro-2-hydroxypropyl)trimethylammonium chloride (DIQUAT chloride);
(ii) about 0.1 wt. % to 30 wt. % of at least one anionic monomer selected from the group consisting of (a) acrylic acid (AA), (b) acrylamidomethylpropyl sulfonate (AMPS), and/or (c) sodium methyl allyl sulfonate (SMAS); and
(iii) about 0.1 wt. % to 20.0 wt. % of at least one monomer comprising at least one functionalized or unfunctionalized acryloyl moiety and/or at least one lactam moiety; and
B. about 0.1 wt. % to 50.0 wt. % of at least one cosmetically acceptable excipient.

2. The composition according to claim 1 wherein said composition is a “2-in-1” hair care conditioning shampoo composition capable of providing effective cleaning and conditioning.

3. The composition according to claim 1 wherein said monomer having at least one functionalized or unfunctionalized acryloyl moiety and at least one lactam moiety has the structure: 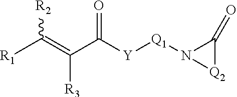
wherein each R₁, R₂ and R₃ is independently selected from the group consisting of hydrogen, halogens, functionalized and unfunctionalized C₁-C₄ alkyl, and 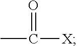
each X is independently selected from the group consisting of OR₄, OM, halogen, N(R₅)(R₆), 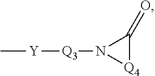
and combinations thereof;
each Y is independently oxygen, NR₇ or sulfur;
each R₄, R₅, R₆ and R₇ is independently selected from the group consisting of hydrogen and functionalized and unfunctionalized alkyl;
each M is independently selected from the group consisting of metal ions, ammonium ions, organic ammonium cations, and combinations thereof; and
each Q₁, Q₂, Q₃, and Q₄ is independently selected from the group consisting of functionalized and unfunctionalized alkylene.

4. The composition according to claim 3 wherein each said Q₁, Q₂, Q₃, and Q₄ is independently selected from the group consisting of functionalized and unfunctionalized C₁-C₁₂ alkylene.

5. The composition according to claim 3 wherein each said R₁, R₂ and R₃ is independently selected from the group consisting of hydrogen, methyl and combinations thereof.

6. The composition according to claim 3 wherein each said R₁ and R₃ is independently hydrogen or methyl; said R₂ is 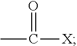
X is selected from the group consisting of OR₄, OM, halogens, and N(R₅)(R₆); each R₄, R₅, and R₆ is independently selected from the group consisting of hydrogen and functionalized and unfunctionalized alkyl; and each M is independently selected from the group consisting of metal ions, ammonium ions, organic ammonium cations, and combinations thereof.

7. The composition according to claim 3 wherein said monomer having at least one functionalized or unfunctionalized acryloyl moiety and at least one lactam moiety has a structure selected from the group consisting of: 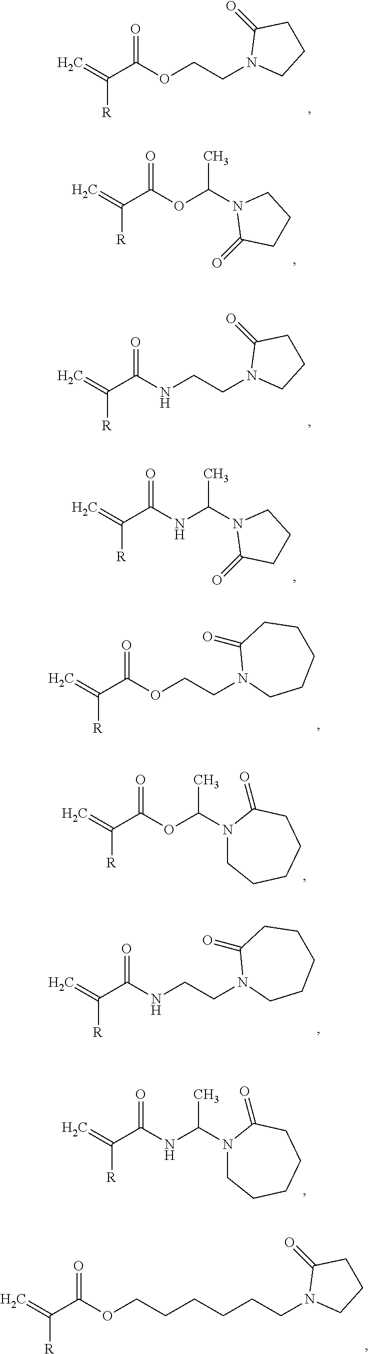 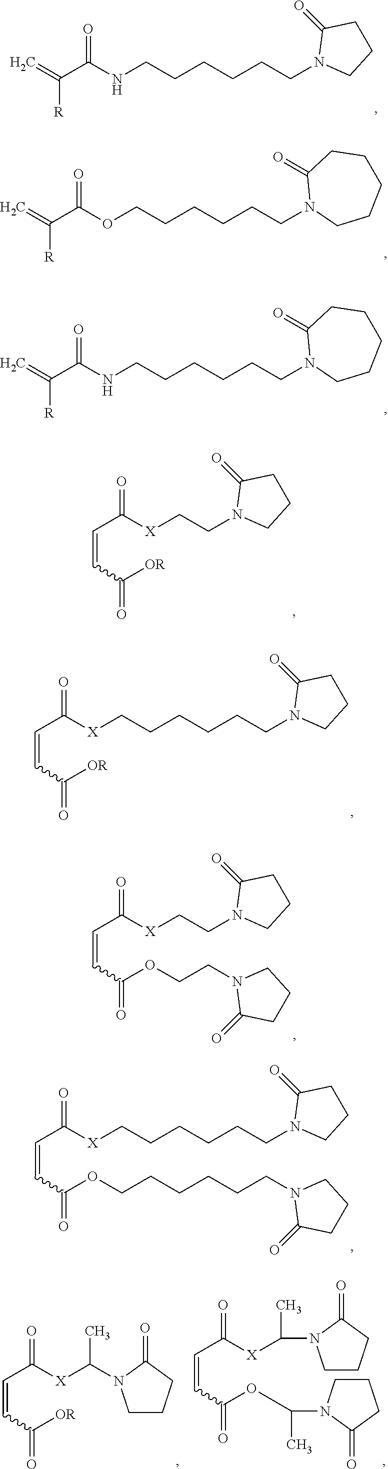 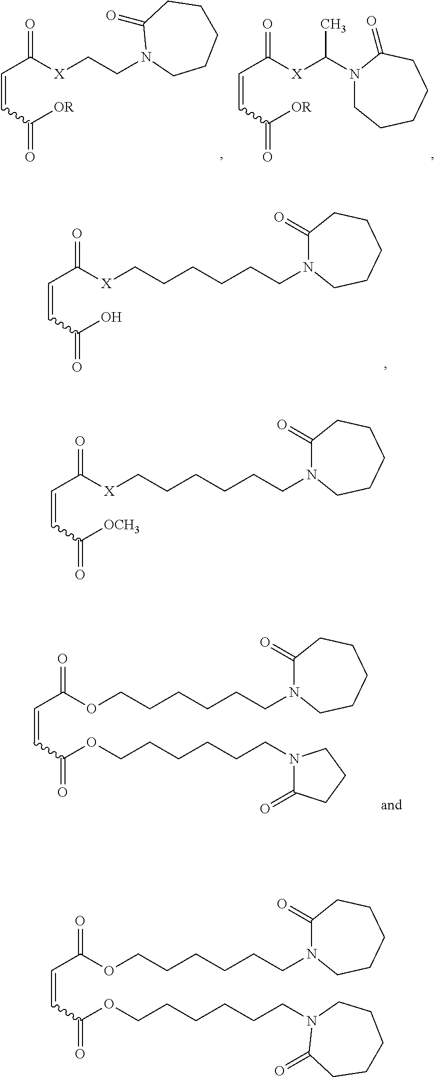
wherein R=H, CH₃ and X=O, NH.

8. The composition according to claim 1, wherein said conditioning terpolymer has a structure selected from the group consisting of:

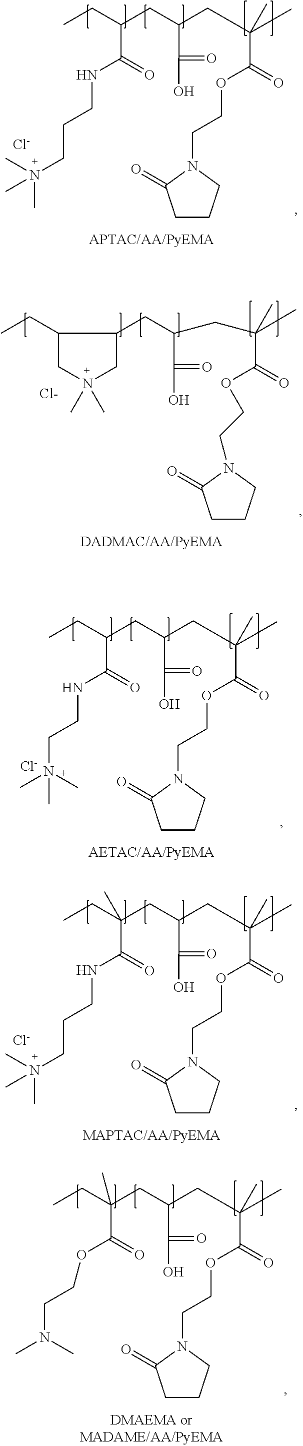

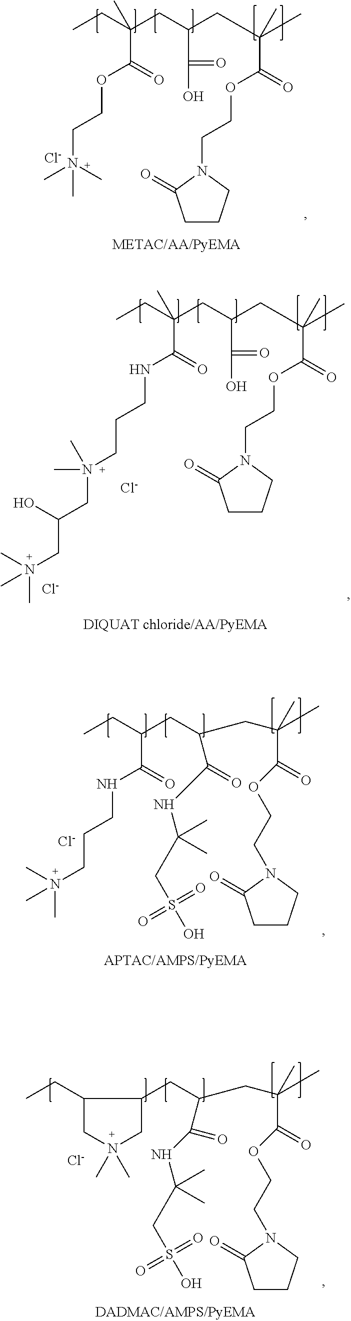

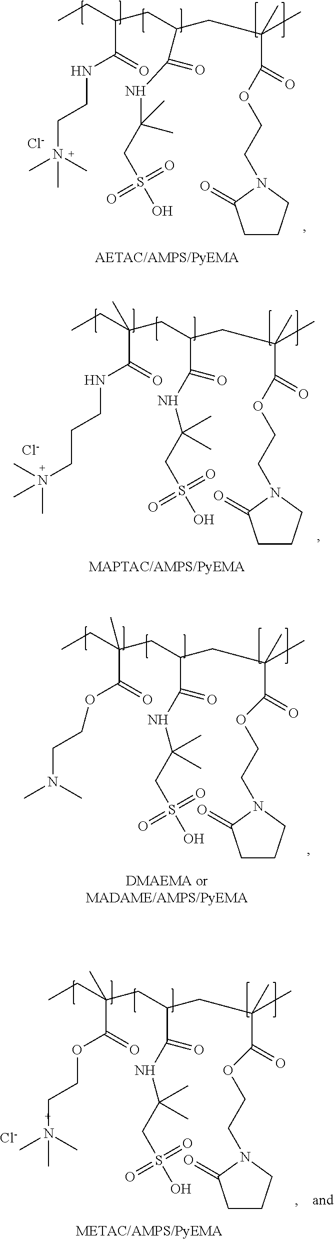

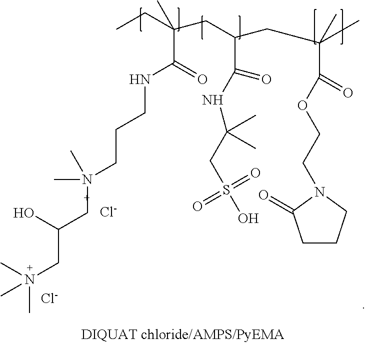

9. The composition according to claim 1 wherein said conditioning polymer further comprises repeating units derived from at least one monomer selected from the group consisting of functionalized and unfunctionalized N-vinyl lactams, N-vinyl-2-pyrrolidone, N-Vinylcaprolactam, alkyl vinyl ethers, methyl vinyl ether, isobutyl vinyl ether, vinyl alkanoates, vinyl acetate, vinyl alkanamides, vinyl alcohols, vinyl carbonates, vinyl carbamates, vinyl thiocarbamates, vinyl ureas, vinyl halides, vinyl imidazoles, vinyl pyridines, vinyl silanes, vinyl siloxanes, vinyl sulfones, maleic anhydride, maleates, fumarates, maleimides, maleamic acids, alpha-olefins, isobutylene, vinyl triazoles, alpha, beta-olefinically unsaturated carboxylic nitriles, acrylonitrile, styrenes, and combinations thereof.

10. The composition according to claim 1, wherein the average molecular weight of said terpolymer is in the range of from about 1000 to 5000,000 g/mol as determined by gel permeation chromatography.

11. The composition according to claim 1, wherein said terpolymer has a cationic degree of substitution (Cat-DS) of greater than about 0.001 units and cationic charge density of about 1 meq/g to about 6 meq/g.

12. The composition according to claim 1, wherein the composition is capable of treating hair conditioning properties comprising detangling, wet combability, wet feel, dry combability, dry feel, sheen, static flyaway control, hydrophobicity, surface smoothening, improved deposition, and no build-up.

13. The composition according to claim 1, wherein the pH of said hair care shampoo composition is in the range of from about 3 to about 13.

14. The composition according to claim 1, wherein said cosmetically acceptable excipient is selected from the group consisting of fatty substances, gelling agents, thickeners, surfactants, moisturizers, emollients, hydrophilic or lipophilic active agents, antioxidants, sequestering agents, preserving agents, acidifying or basifying agents, fragrances, fillers, dyestuffs, emulsifying agents, solvents, UV-A or UV-B blocker/filters, plant extracts, moisturizers, proteins, peptides, neutralizing agents, solvents, silicones and/or reducing agents.

15. A method for providing reduced squeakiness effect for hair substrate or keratin fibers comprising the steps of:
i. wetting the hair with water;
ii. applying or contacting an effective amount of the shampoo composition of claim 1 to the wet hair substrate or keratin fibers of step (i); and
iii. rinsing off the composition from the hair using water.

16. A process for preparing a conditioning terpolymer of claim 1 comprising polymerizing:
i. about 50 wt. % to 99.9 wt. % of at least one cationic or pseudo-cationic monomer selected from the group consisting of acrylamidopropyl trimethylammonium chloride (APTAC), diallyl dimethyl ammonium chloride (DADMAC); Acryloyloxyethyltrimethylammonium chloride (AETAC); Methacrylamidopropyltrimethylammonium chloride (MAPTAC); Dimethylaminoethyl methacrylate (DMAEMA or MADAME); Methyloyloxyethyl trimethyl ammonium chloride (METAC); DimethylaminopropylmethacrylamideN-(3-chloro-2-hydroxypropyl)trimethylammonium chloride (DIQUAT chloride);
ii. about 0.1 wt. % to 30 wt. % of at least one anionic monomer selected from the group consisting of (a) acrylic acid (AA), (b) acrylamidomethylpropyl sulfonate (AMPS), and/or (c) sodium methyl allyl sulfonate (SMAS); and
iii. about 0.1 wt. % to 20.0 wt. % of at least one monomer comprising at least one functionalized or unfunctionalized acryloyl moiety and at least one lactam moiety;
said terpolymer having a cationic degree of substitution (Cat-DS) of greater than about 0.001 units, and wherein the cationic charge density is in the range of about 1 meq/g to about 6 meq/g.

17. The process according to claim 15, wherein the terpolymer is prepared by radical polymerization, emulsion polymerization, ionic chain polymerization, bulk polymerization, suspension polymerization or precipitation polymerization.

18. The process according to claim 15, wherein the average molecular weight of the terpolymer is in the range of from about 100,000 to 1,000,000 g/mol as determined by gel permeation chromatography.

19. A method of cleansing and conditioning hair using the shampoo composition of claim 1 comprising the steps of:
i. wetting the hair with water;
ii. applying an effective amount of the shampoo composition of claim 1 to the wet hair of step (i); and
iii. rinsing off the composition from the hair using water.
